(19) United States
(12) Patent Application Publication
(10) Pub. No.: US 2006/0251208 A1
(43) Pub. Date: Nov. 9, 2006

(54) Method for reconstructing a radiographic image by combining elemental images

(76) Inventors: Christine Robert-Coutant, Saint-Martin D'Uriage (FR); Georges Gonon, Claix (FR); Jean-Marc Dinten, Lyon (FR)

Correspondence Address: Thelen Reid & Priest, PO Box 640640, San Jose, CA, 95164-0640, US

(21) Appl. No.: 10/540,069

(22) PCT Filed: Dec. 19, 2003
(86) PCT No.: PCT/FR03/50195

§ 371 Date: Jun. 30, 2006

(30) Foreign Application Priority Data
Dec. 23, 2002 (FR) 02 16538

(51) Int. Cl. H05G 1/60; A61B 6/00; G01N 23/00; G21K 1/12

(52) U.S. Cl. 378/4; 378/901

(57) ABSTRACT

In a radiographic method where an object (2) is studied in several steps by a radiation (4) occupying various positions (4i, 4j) just as the associated network of detectors (3i, 3j), the combination of elementary vignettes into an overall image without junction defects is resolved by a discretisation of the object (2) into volumes and a calculation of the attenuation in each volume (voxel) (8) to obtain images of the object at different reconstruction heights; then said images are combined with each other to obtain a final, more exact image.

The invention concerns a method for reconstructing a radiographic image by combination of a collection of overlapping vignettes.

An important application of the invention, but which is not exclusive, is osteodensitometry, in other words the measurement of the bone mineral density (BMD) of the body, where measurements are also taken of the composition of the tissues while distinguishing the non-fatty from the fatty tissues. The examination may concern extensive regions of the body. BMD is expressed as a mass per unit of surface, which corresponds to the projection, along parallel lines, of the bone mass on a plane relating to a unit surface. Multiplying the BMD by the surface of the bone gives the bone mineral content or BMC. An interest of the invention in this application will be not only to provide better images, as we will describe hereafter, but more accurate specific measurements.

In radiography, large sized images are frequently obtained by bits, by means of projection vignettes that are taken successively by displacing the radiation passing through the object to different positions, as well as the mono-dimensional or two-dimensional network of detectors taking the measurements. The assembling of the vignettes then gives the desired image.

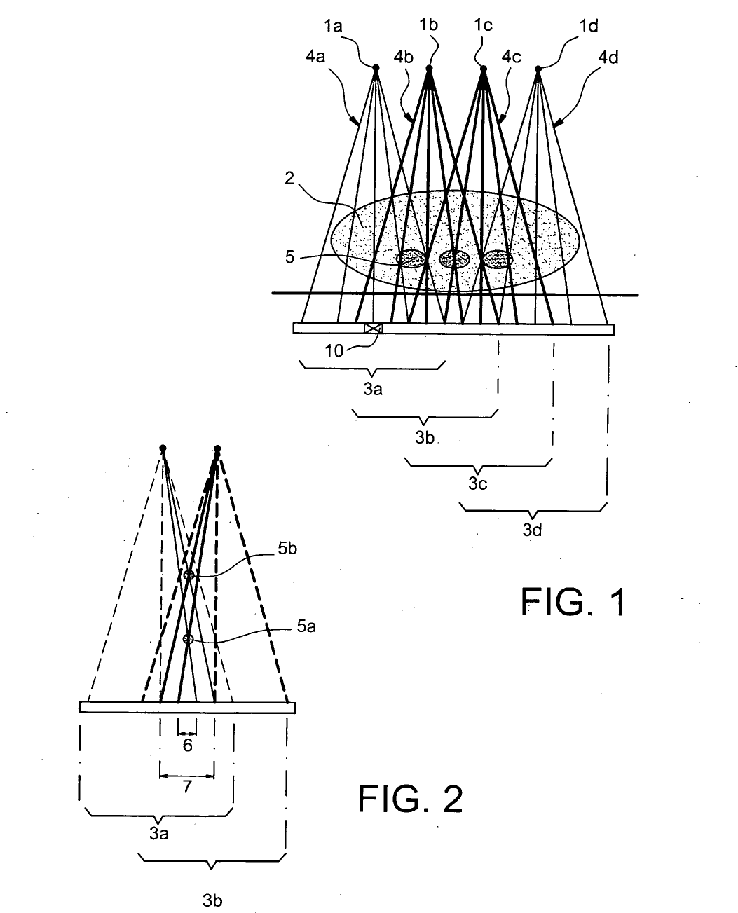

The method becomes complicated in the normal case of a divergent radiation from a focal point towards the network of detectors, either in a cone or in a series of plane and parallel fans. FIG. 1 shows the normal measurement configuration: the radiation comprises a source 1 (irregular or linear) that one displaces at each measurement along the object 2 as well as the network of detectors 3. The positions taken are noted 1a, 1b, 1c, 1d, and 3a, 3b, 3c and 3d. In order for the attenuation of the radiation to be measured at any locality of the object 2, the radiation beam 4 must comprise overlap portions, sufficiently wide so that each point of the object 2 is seen at least once, in the positions 4a, 4b, 4c and 4d that one makes it take, and the projection vignettes of the radiation, the positions of which coincide with that of 3a, 3b, 3c and 3d that the network of detectors 3 takes successively, have likewise portions of overlap. It is therefore impossible to simply juxtapose the vignettes to obtain the overall image of the object, but one must, on the contrary, determine the positions of the portions of overlap on the vignettes and carry out a synthesis of the contents of said portions of overlap to reconstruct the image.

Another problem that appears is that the magnification of details depends on their distance from the source 1. The projection width of details 5 of the object 2 on the network of detectors 3 will be proportionally wider if the details 5 are nearer the source 1. Thus, a divergent radiation does not enable the vignettes to be juxtaposed easily, nor to respect the scale of details within each vignette.

FIG. 2 enables these problems to be explained. Two details 5a and 5b spaced vertically are located in the portion of overlap of the vignettes taken by the network of detector 3 at the positions 3a and 3b. The rays crossing through the detail 5a are distant from each other by the gap 6 on the plane of the network of detectors 3, and those that pass through the detail 5b are distant from each other by the gap 7 on the same plane; the gaps are different to each other, and different from the displacement that needed to be imposed on the network of detectors 3 between the positions 3a and 3b where the views have been taken. A good reconstruction of the image at the portions of overlap makes it necessary to combine between them the measurements associated with each of the details for the different vignettes, which is impossible to do directly since their heights are in general unknown. If one chooses for example to associate the rays distant from each other by the gap 6 to reconstruct the portions of overlap, the details at the level of 5a will be captured correctly, but the details present at other heights cannot be. The combination of the vignettes will then produce blurredness and an inaccurate magnification of these other details.

However, a method of this type has already been proposed in the prior art. The image is reconstructed by choosing an exact reconstruction of the portions of overlap at determined heights, in those places where important details, and particularly the bones for a radiography of the body, are likely to be located. In order to obtain good results, the height of these details therefore first needs to be known. Weighting coefficients may favour the results of one or another of the vignettes depending on the considered position on the portion of overlap. The restitution of the other details of the image is sacrificed.

Another known method consists in calculating the correlations between the portions of overlap of the different vignettes in order to evaluate the gap (6, 7 or other) of the rays to be associated to carry out the synthesis of the portions of overlap. The correlations depend on the prominent details present on the two portions of overlap and stemming from a same location of the object 2. The reconstruction of the image is accomplished at the level of said prominent details and it is good, if at least said details exist; however, as in the previous method, the details located at the other heights will be poorly captured.

It should be added that height conflicts may appear if the portions of overlap are numerous, and in particular with a conic radiation where the portions of overlap concern the whole perimeter of the vignettes. Two portions of overlap on two sides of a vignette could be reconstructed independently at different heights, while at the same time having an intersection for which it will be awkward to choose a reconstruction height.

A more accurate method of reconstructing a radiographic image is proposed with the invention. It is based on a general discretisation of the object into volumes (voxels) defining reconstruction heights, and the combinations of the attenuation values estimated on each of the volumes at the different reconstruction heights in order to improve the overall image, without necessarily favouring one reconstruction height.

In a more detailed manner, the invention generally concerns a method for reconstructing a radiographic image from an object crossed by a divergent radiation undergoing an attenuation, the radiation occupying successive positions having overlapping portions and the attenuation being measured by a network of detectors, on which the radiation projects and giving vignettes of the image respectively associated with the positions of the radiation and thus comprising overlapping portions, the method comprising a combination of vignettes for reconstructing the image, as well as the following steps:

discretising the object into voxels defining reconstruction heights,

associating each voxel with at least one detector respective of the network on which the radiation projects after having crossed said voxel,

allocating an attenuation value to each voxel according to the values measured by said associated detector,

and combining the attenuation values of the voxels at the different reconstruction heights in order to obtain a two dimensional image.

In one of the forms of the invention, the attenuation value attributed to each voxels is equal to the sum of the values measured by said associated detector, divided by the number of vignettes that contribute to giving said associated detector and by the length of each voxel that has been crossed, and the attenuation values of the voxels are combined by a digital combination on the groups of voxels superimposed at the different reconstruction heights. And, in another of its forms, the attenuation value attributed to each voxel is obtained by iterative projection of the attenuation values measured by the detectors, provisional values being allocated to the voxels and corrected after having been projected on the detectors, by calculating the differences between the sums and the provisional values on the projection lines at the values measured by the detectors on said projection lines, and by spreading out the differences on said projection lines to correct the provisional values. One also carries out a digital combination of the groups of voxels.

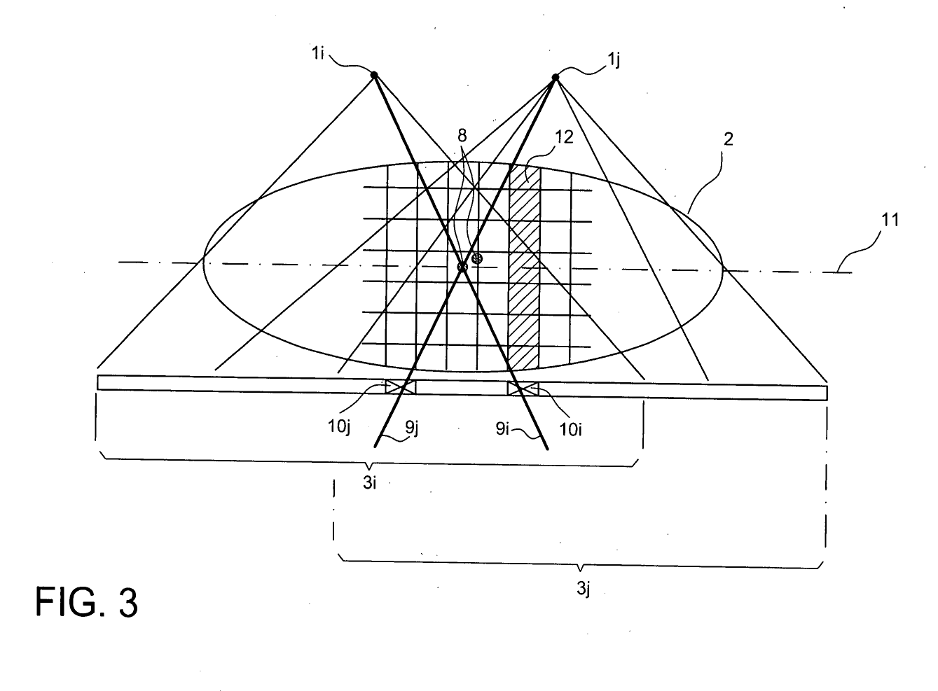

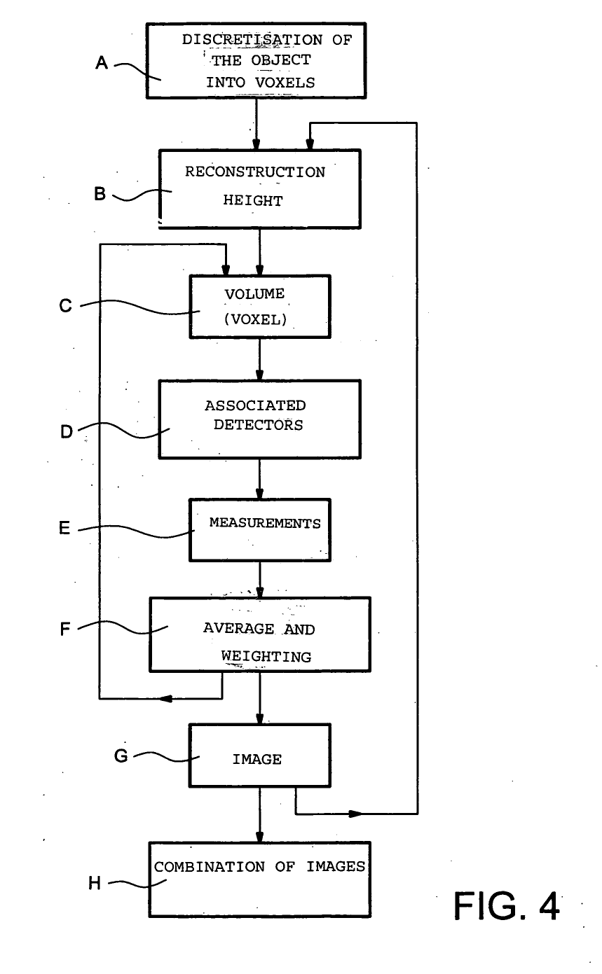

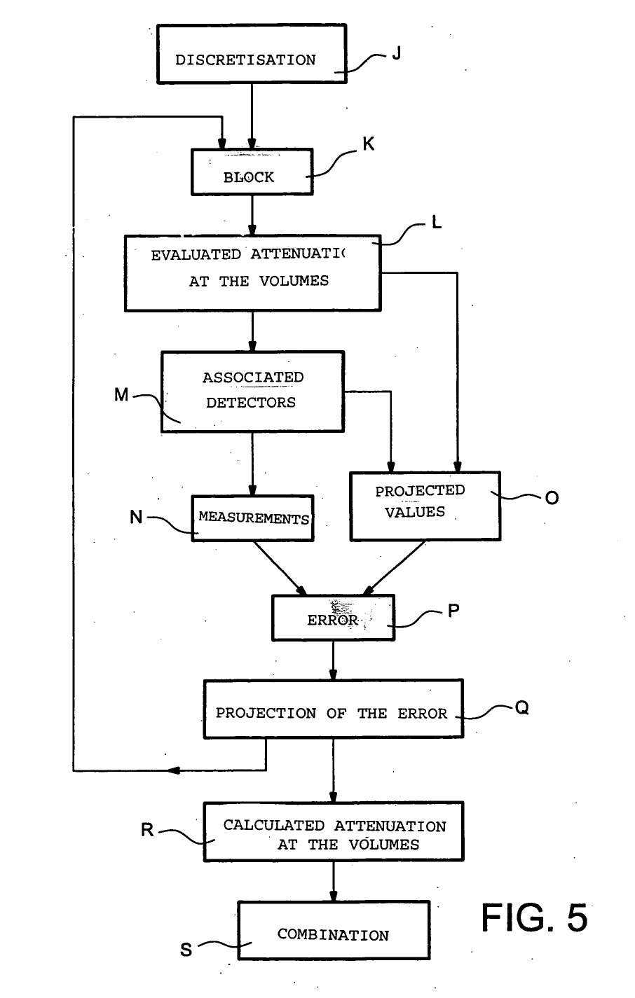

The invention will now be completely described in relation to the figures, in which FIG. 1 schematises the method of forming the vignettes, FIG. 2 illustrates the problem of reconstruction at an arbitrary height, FIG. 3 illustrates the explanatory elements of the invention, and FIGS. 4 and 5 are flow charts of two modes of the method.

We will now comment on FIG. 3. The object 2 is discretised into elementary volumes or voxels 8 that define reconstruction heights 11. The radiation passes through the voxels 8 by the rays 9i and 9j, which are several for the voxels 8 belonging to the portions of overlap, and which originate from the respective positions 1i and 1j of the source 1 and project on the respective detectors 10i and 10j that are associated with them for the corresponding positions 3i and 3j of the network of detectors 3. The detectors 10 measure the attenuations of the rays 9 through the whole object 2, and therefore through all the voxels such as 8 that they cross. In practice, the voxels 8 project on a surface that may encompass several detectors 10 completely, and others partially. The system is calibrated to associate with each voxel 8 the detectors 10 on which it projects and share out between them the proportions of its attenuation. In order to simplify the explanations, we will not evoke here these calibration techniques, which are completely usual in the art, and we will consider voxels 8 that project completely on a unique detector 10 along a unique projection ray.

According to FIG. 4, the method begins by a step A of general discretisation of the object 2 into voxels 8, the layers of which define the reconstruction heights 11 of the image. In practice, the reconstruction heights 11 will be quite limited in number and the voxels 8 more parallelepipedal, lengthened in height, than cubic. The following steps B and C consist in placing oneself at a determined reconstruction height 11 and a voxel 8. One then searches for the rays such that 9i and 9j crossing through the voxel 8 considered, and the detectors 10i and 10j of projection of said rays on the network of detectors 3, in step D. The following step E consists in reading the measurement of the attenuation of the rays 9i and 9j on the detectors 10i and 10j. In the following step F, an average of these attenuations is worked out, at least for the voxels 8 belonging to the portions of overlap and which are thus crossed by at least two rays 9. A weighting may be applied to the different measures, by according for example more weight to that which comes from the substantially vertical rays, which improves in particular the image at the positions of overlap of the vignettes. The steps C to E or F are then repeated for all the volumes of the considered layer; after which, at the step G, an image of the object 2 is reconstructed.

This image is an image of the whole of the object 2, and not only a sectional image at the considered height, since the attenuations measured by the detectors 10 along the rays 9 have been assumed to be concentrated at the voxels 8 of the layer at this height.

Then, one returns to step B to reconstruct the object 2 at another height, and the cycle of steps C to G restarts with the voxels 8 of the associated layer.

When the images of the object 2 have been reconstructed at all of the heights, they are combined at the step H with the hope of obtaining a more exact image. Several methods may be envisaged. The simplest consists perhaps in averaging the images on the columns 12 (in FIG. 3) of stacked voxels 8 belonging to different layers, with if necessary a weighting to favour the most representatives layers. If necessary, one may choose just one of the images that one considers better than the others, or an assembly of several images at the locations that they best represent. All of these methods should give better results than those of the prior art that we have described previously.

We will only mention certain correction methods that are normal in the art and which are not affected by the invention.

The diffused radiation may firstly be subtracted from the measurements before these are exploited. Several methods exist for carrying out this subtraction, the most simple of which, given by way of example, is perhaps to carry out an additional measurement where a screen is intercalated between the object 2 and the network of detectors 3 while masking certain of the detectors 10. The masked detectors 10 are not touched by the direct radiation of the rays 9, but only by the diffused radiation, which is then measured by these detectors and which may be deduced by interpolations for the other detectors.

The attenuations of a radiation may in general be expressed by a multiplication coefficient of the initial radiation Io less than unity and equal to ₑ−μl where l is the attenuation length and μ the attenuation coefficient characteristic of the material, and which is in general the value that one seeks to reconstruct the image. The detectors 10 directly measure the radiation I that has not been absorbed by the object 2 and which is equal to Iₒ e^{-μl}; one may deduct from this the product μl, then the value of μ if one divides the values of μl by the crossing lengths of the object 2 by the rays 9, after having estimated them by another measurement or having evaluated them geometrically.

Another embodiment of the invention will now be described by means of FIG. 5. After a step of discretisation J similar to that A of the previous embodiment, one carries out at best a division into blocks at step K. Indeed, the resolution that is going to be undertaken may become difficult if the considered system is too voluminous. In practice, each block could comprise the voxels 8 associated with a vignette. Whether a division into blocks is carried out or not, the problem to be solved may be expressed as \vec{p}=M\vec{x} where \vec{x} designates the unknowns, in other words the attenuations at the voxels 8, \vec{p} designates the projections of these values, in other words the measurements by the detectors 10, and finally M designates the matrix of projection. The coefficients mᵢⱼ of the matrix M represent the contribution of a voxel 8 of index j at the projection following the ray 9 of index i, and may in general be approached by the length crossed by said ray within this volume.

The following step L is an evaluation of the attenuation at the voxels 8 of the considered block. The first evaluation may be arbitrary, for example at zero values. For each of the voxels 8, one searches for, according to step M, the detector 10 that is associated with it by the ray 9 that crosses it, as in step D of the previous embodiment. The following step N is a reading of the measurements of the detectors 10 similar to the step E. The determination of the projection rays 9 make it possible to perform an evaluation of the projected attenuation values at step O, in other words that one performs the calculation M\vec{x} in order to evaluate \vec{p}. By subtracting these evaluated values for the projections from the real, measured values of the same projections, one determines the error made in the evaluation of the projected values at step P.

The following step Q is a projection of said error in the voxels 8 of the object 2 in order to correct the evaluation values of the attenuation. In concrete terms, one proceeds by applying the formula

$${{x}^{->}}^{( q + 1 )} = {{x}^{->}}^{( q )} + {λ}^{( q )} \frac{{}^{t} {M}_{bloc}}{{‖}^{t} {M}_{bloc} \cdot {M}_{bloc} ‖} ( {{P}^{->}}_{bloc} - {M}_{bloc} \cdot {{x}^{->}}^{( q )} )$$

\vec{x}^{(q)} are successive evaluations of the attenuation at the voxels 8 of block; λ^{(q)} is a relaxation coefficient that makes it possible not to head too quickly towards a solution that only corresponds to the first blocks and which is between 0 and 2; moreover, said coefficient is not uniform in the blocks but may advantageously be higher for the rays that are substantially vertical, or perpendicular to the detectors 10, in order to give them a greater weighting importance, as in the previous embodiment; ^{t}M_{bloc} is the transpose of the matrix M for the considered block; the denominator term is a normalisation term; finally, the terms in brackets represent the error calculated at step P.

One proceeds in the same way for the following block, by restarting the cycle from step K to step Q, then one returns to the first block for a new iteration, until the evaluated attenuations have converged towards a solution, which one expresses by the step R. The voxels 8 included in the portions of overlap of the object 2 have been treated in the same way as the others, simply undergoing more iterations, for each of the blocks to which they belong.

One then disposes of a three dimensional image of the object 2; a two dimensional image of good quality may be obtained by a combination of the values obtained, which consists in combining the attenuation values on the columns 12 of stacked volumes 8.

The method of the invention makes it possible to reconcile a good quality of restitution of important details of the studied object with a good overall quality of the image. It is possible to obtain images in which the resolution is analogous to the pitch of the detectors 10.

One is placed in the normal situation where the network 3 of detectors accompanies the movement of the radiation 4, but the method could be applied with change with a network of detectors immobile under the object 2 and the surface of which would extend and to all of the projection vignettes.

1. Method for reconstructing a radiographic image of an object crossed by a diverging radiation undergoing an attenuation, the radiation occupying successive positions having overlapping portions and the attenuation being measured by a network of detectors, on which the radiation projects and giving vignettes of the image respectively associated with the positions of the radiation and also comprising overlapping portions, the method comprising a combination of vignettes for reconstructing the image, as well as the following steps:
discretising the object into voxels defining reconstruction heights,
associating the voxels with at least one detector respective of the network on which the radiation projects after having crossed said volume,
allocating an attenuation value to each voxel according to the values measured by said associated detector,
and combining the attenuation values of the voxels at the different reconstruction heights to obtain a two dimensional image.

2. Method for reconstructing a radiographic image according to claim 1, characterised in that the attenuation value attributed to each volume is equal to the sum of the values measured by said associated detector, divided by the number of vignettes that contribute to giving said associated detector, and the attenuation values of the voxels are combined by a digital combination on the groups of voxels superimposed at the different reconstruction heights.

3. Method for reconstructing a radiographic image according to claim 1, characterised in that the attenuation value attributed to each voxel is obtained by iterative projection of attenuation values measured by the detectors, provisional values being allocated to the voxels and corrected after having been projected on the detectors, in calculating the differences between the sums of provisional values on the projection lines and the values measured by the detectors on said projection lines, and by projecting the differences on said projection lines to correct the provisional values.

4. Method for reconstructing a radiographic image according to claim 2, characterised in that the attenuation values of the volumes are digitally combined on the groups of volumes superimposed at the different reconstruction heights.

5. Method for reconstructing a radiographic image according to claim 1, characterised in that it is applied to osteodensitometry.

6. Method for reconstructing a radiographic image according to claim 3, characterised in that the attenuation values of the volumes are digitally combined on the groups of volumes superimposed at the different reconstruction heights.
